(19) United States
(12) Patent Application Publication
(10) Pub. No.: US 2026/0002350 A1
(43) Pub. Date: Jan. 1, 2026

(54) TOILET SYSTEM

(71) Applicant: TOTO LTD., Kitakyushu-shi (JP)

(72) Inventors: Kenta SUZUKI, Kitakyushu-shi (JP); Tetsuya UCHIDA, Kitakyushu-shi (JP); Kaori KUWAHARA, Kitakyushu-shi (JP); Ryota TAKAHASHI, Kitakyushu-shi (JP)

(21) Appl. No.: 19/319,561

(22) Filed: Sep. 4, 2025

Related U.S. Application Data
(63) Continuation of application No. PCT/JP2024/002271, filed on Jan. 25, 2024.

(30) Foreign Application Priority Data
Mar. 31, 2023 (JP) 2023-057688

(51) Int. Cl. E03D 9/00; A61B 5/00; A61B 5/026; G16H 40/63

(52) U.S. Cl. CPC E03D 9/00; A61B 5/0261; A61B 5/7246; G16H 40/63

(57) ABSTRACT

The present invention is a toilet system including: a toilet seat having a seat surface on which a user is to sit; a sensor configured to measure a physical quantity which reflects blood flow information of the user; a fitness level evaluation part configured to evaluate a fitness level of the user based on a measurement result of the sensor; and a fitness level output part configured to output the fitness level of the user which has been evaluated by the fitness level evaluation part; wherein the fitness level evaluation part is configured to evaluate the fitness level of the user based on the measurement result of the sensor that has been accumulated for one or more time periods that satisfy a predetermined condition up to a predetermined total time period.

### CROSS-REFERENCE TO RELATED APPLICATIONS

This application is a Continuation of International Application No. PCT/JP2024/002271, filed Jan. 25, 2024, which claims priority to Japanese Application No. JP 2023-057688, filed Mar. 31, 2025, the disclosures of which are hereby incorporated by reference in their entirety.

### TECHNICAL FIELD

The present invention pertains to a toilet system that can provide an evaluation result of fitness level as one of health indices of a user.

### BACKGROUND ART

JP-A-2021-68396 (Patent Document 1) has disclosed a system that measures blood flow conditions of a toilet user who sits on a toilet seat, calculates a health index based on measurement results thereof, and outputs the calculated health index (for example, causes a display terminal to display the calculated health index).

JP-A-2016-32579 (Patent Document 2) has disclosed a wearable device that evaluates athletic ability of a subject based on a pulse rate of the subject while the subject exercises under a state wherein the subject wears an inertial sensor and a pulse meter. In this device, a prerequisite for measuring the pulse rate (and thus for evaluating the athletic ability) is that the subject exercises.

### Prior Art Document List

### Patent Document

[Patent Document 1] JP-A-2021-68396
[Patent Document 2] JP-A-2016-32579

### SUMMARY OF INVENTION

### Technical Problem to be Solved by Invention

As described above, it is already possible to calculate (determine, diagnose) a plurality of health indices of a user, from blood flow conditions of the user under a state wherein the user sits on a toilet seat.

Specifically, based on a Dynamic Light Scattering method, a laser sensor is attached in or on a toilet seat, which can measure blood flow conditions within a skin on a back side of a user's thigh. A Fourier transform or the like is applied to an output signal from the laser sensor so that a plurality of health indices (including a pulse wave, a blood flow amount, a heart rate, or the like) can be calculated (derived).

FIG. 6 shows an example of an output signal from a laser sensor. This is a signal by a laser Doppler blood flow measurement method and represents an oscillation in skin blood flow perfusion (peripheral blood flow).

FIG. 7 shows an example of a graph obtained by performing a wavelet transform on the signal shown in FIG. 6. Neurogenic activity of the vascular endothelium appears in frequency domain A(0.0095-0.021 Hz), neurogenic activity of the vascular wall appears in frequency domain B (0.021-0.052 Hz), neurogenic activity of the vascular smooth muscle appears in frequency domain C (0.052-0.145 Hz), respiratory activity appears in frequency domain D (0.145-0.6 Hz), and cardiac activity appears in frequency domain E (0.6-2 Hz).

Herein, an amount of the output signal from the laser sensor (i.e. a measurement time by the laser sensor) required to calculate a health index with a certain degree of accuracy (reliability) may be different for each health index.

For example, with respect to a “heart rate”, a calculated value thereof with a certain degree of accuracy (reliability) can be obtained from an output waveform signal corresponding to 10 seconds (as a measurement time). On the other hand, with respect to a “vascular age” and a “relaxation level (stress state)”, it is required to use an output waveform signal corresponding to 60 seconds (as a measurement time) to obtain a calculated value thereof with a certain degree of accuracy (reliability).

Among various health indices, the present invention focuses on fitness level. The fitness level is an index of cardiopulmonary capacity. The fitness level is known to correlate with a maximum oxygen uptake (VO2 Max), which is a maximum amount of oxygen that a person can take into his or her body per minute, which means a maximum amount of oxygen that his or her body can consume per minute during exercise, but also correlates with a blood information signal and its change (e.g., correlates with a change in a heart rate and/or in an amount of blood flow). Therefore, based on the strength of such a signal and its change, the fitness level can be estimated and evaluated.

Then, the inventors have found that, when evaluating the fitness level, accumulating (collecting) measurement results for time periods that satisfy a predetermined condition up to a predetermined total time period will lead to a result that more accurately reflects a user's actual fitness level.

More specifically, the predetermined condition may be that a signal for determining a stable fluctuation based on the measurement signal of the sensor has once entered a stable fluctuation state (in this case, measurement data from a continuous time period from when the signal for determining the stable fluctuation has once entered the stable fluctuation state until the predetermined total time period has elapsed can be utilized).

Alternatively, the predetermined condition may be that a signal for determining a stable fluctuation based on the measurement signal of the sensor maintains a stable fluctuation state (in this case, measurement data from one or more time periods during which the stable fluctuation state is maintained can be accumulated (joined together if discontinuous) until the predetermined total time period has elapsed, and can be utilized).

Alternatively, the predetermined condition may be that a user has been seated on the toilet seat for more than a predetermined sitting time and a signal for determining a stable fluctuation based on the measurement signal of the sensor maintains a stable fluctuation state (in this case, for example, determination as to whether the predetermined sitting time has elapsed is used as a substitute for determination as to whether the stable fluctuation state has been once entered, and subsequently measurement data from one or more time periods during which the stable fluctuation state is maintained can be accumulated (joined together if discontinuous) until the predetermined total time period has elapsed, and can be utilized).

The present invention has been made based on the above findings by the inventors. The object of the present invention is to provide a toilet system that can provide an evaluation result of fitness level of a user and that can provide an evaluation result that more accurately reflects actual fitness level of the user.

### Means for Solving Problem

The present invention is a toilet system including: a toilet seat having a seat surface on which a user is to sit; a sensor configured to measure a physical quantity which reflects blood flow information of the user; a fitness level evaluation part configured to evaluate a fitness level of the user based on a measurement result of the sensor; and a fitness level output part configured to output the fitness level of the user which has been evaluated by the fitness level evaluation part; wherein the fitness level evaluation part is configured to evaluate the fitness level of the user based on the measurement result of the sensor that has been accumulated for one or more time periods that satisfy a predetermined condition up to a predetermined total time period.

According to the present invention, by setting the “predetermined condition” and the “predetermined total time period” appropriately for evaluating the fitness level, it is possible to provide an evaluation result that more accurately reflects actual fitness level of the user.

The predetermined total time period may be between 15 seconds and 60 seconds, preferably between 30 seconds and 60 seconds, and more preferably 45 seconds.

The predetermined condition may be that a signal for determining a stable fluctuation based on the measurement signal of the sensor (for example, a signal processed to represent a pulse wave is used) has once entered a stable fluctuation state (for example, the user's pulse wave has once entered a stable fluctuation state). In this case, measurement data from a continuous time period from when the signal for determining the stable fluctuation has once entered the stable fluctuation state until the predetermined total time period has elapsed can be utilized.

According to the above feature, measurement data from a time period before the signal for determining the stable fluctuation has once entered the stable fluctuation state can be excluded from the evaluation target. Therefore, it is possible to provide an evaluation result that more accurately reflects actual fitness level of the user.

Alternatively, the predetermined condition may be that a signal for determining a stable fluctuation based on the measurement signal of the sensor (for example, a signal processed to represent a pulse wave is used) maintains a stable fluctuation state (for example, the user's pulse wave maintains a stable fluctuation state). in this case, measurement data from one or more time periods during which the stable fluctuation state is maintained can be accumulated (joined together if discontinuous) until the predetermined total time period has elapsed, and can be utilized).

According to the above feature, measurement data from a time period before the signal for determining the stable fluctuation has first entered the stable fluctuation state can be excluded from the evaluation target. In addition, measurement data from one or more time periods during which the signal for determining the stable fluctuation deviates from the stable fluctuation state, which may be caused by the user's “straining” while using the toilet or by other “body movements”, can be also excluded from the evaluation target. Therefore, it is possible to provide an evaluation result that even more accurately reflects actual fitness level of the user.

Alternatively, the predetermined condition may be that a user has been seated on the toilet seat for more than a predetermined sitting time and a signal for determining a stable fluctuation based on the measurement signal of the sensor (for example, a signal processed to represent a pulse wave is used) maintains a stable fluctuation state (for example, the user's pulse wave maintains a stable fluctuation state). In this case, for example, determination as to whether the predetermined sitting time has elapsed is used as a substitute for determination as to whether the stable fluctuation state has been once entered, and subsequently measurement data from one or more time periods during which the stable fluctuation state is maintained can be accumulated (joined together if discontinuous) until the predetermined total time period has elapsed, and can be utilized.

According to the above feature, determination as to whether the predetermined sitting time has elapsed is used as a substitute for determination as to whether the signal for determining the stable fluctuation has once entered the stable fluctuation state. Therefore, it is possible to simplify a signal state determination routine (program).

Herein, in general, the fitness level is an index of cardiopulmonary capacity which correlates with a change in a heart rate and/or an amount of blood flow of the user.

In addition, in the present invention, it is preferable that the fitness level output part is capable of outputting a comparison result between a fitness level that has been evaluated by the fitness level evaluation part most recently and a fitness level that was evaluated by the fitness level evaluation part in the past.

According to the above feature, a user of the toilet system of the present invention can recognize his or her current fitness level each time the user uses the toilet in daily life while referring to the comparison result with the fitness level in the past.

In addition, in the present invention, it is preferable that the fitness level output part is capable of outputting a comparison result between a fitness level that has been evaluated by the fitness level evaluation part most recently and a predetermined threshold value.

According to the above feature, a user of the toilet system of the present invention can intuitively recognize whether his or her current fitness level is good or not each time the user uses the toilet in daily life. For example, the fitness level may be evaluated as a score value between 0 and 100 (higher values correlate with a state in which the user is training, playing a sport, etc. to give a positive effect while lower values correlate with a state of insufficient exercise, etc.).

In addition, in the present invention, it is preferable that the fitness level output part is configured not to output the fitness level of the user when the fitness level evaluation part does not evaluate the fitness level of the user based on the measurement result of the sensor that has been accumulated for the one or more time periods that satisfy the predetermined condition up to the predetermined total time period.

According to the above feature, it is effectively inhibited that any evaluation result that is likely to incorrectly reflect the user's actual fitness level is outputted as correctly reflecting the user's actual fitness level.

In addition, in the present invention, the sensor may be an optical sensor provided in or on the toilet seat, configured to emit a light toward a leg of the user and to detect a reflected light.

Alternatively, in the present invention, the sensor may be an optical sensor to be fitted onto an index finger of the user who sits on the toilet seat, configured to emit a light toward the index finger of the user and to detect a reflected light.

The present invention should be also protected as a method category.

That is to say, a method according to an aspect of the present invention is a method of providing an evaluation result of fitness level by using a toilet system, the toilet system including: a toilet seat having a seat surface on which a user is to sit; a sensor configured to measure a physical quantity which reflects blood flow information of the user; a fitness level evaluation part configured to evaluate a fitness level of the user based on a measurement result of the sensor; and a fitness level output part configured to output the fitness level of the user which has been evaluated by the fitness level evaluation part; the method including the step of evaluating the fitness level of the user based on the measurement result of the sensor that has been accumulated for one or more time periods that satisfy a predetermined condition up to a predetermined total time period.

The present invention should be also protected as a program category.

That is to say, a program according to an aspect of the present invention is a program for providing an evaluation result of fitness level by using a toilet system, the toilet system including: a toilet seat having a seat surface on which a user is to sit; a sensor configured to measure a physical quantity which reflects blood flow information of the user; a fitness level evaluation part configured to evaluate a fitness level of the user based on a measurement result of the sensor; and a fitness level output part configured to output the fitness level of the user which has been evaluated by the fitness level evaluation part; the program being capable of perform, when executed by a computer, the step of evaluating the fitness level of the user based on the measurement result of the sensor that has been accumulated for one or more time periods that satisfy a predetermined condition up to a predetermined total time period.

### Effects of Invention

According to the present invention, by setting the “predetermined condition” and the “predetermined total time period” appropriately for evaluating the fitness level, it is possible to provide an evaluation result that more accurately reflects actual fitness level of the user.

### BRIEF DESCRIPTION OF DRAWINGS

FIG. 1 is a schematic perspective view of a toilet system according to an embodiment of the present invention,

FIG. 2 is an exploded perspective view of a toilet seat of the toilet system shown in FIG. 1,

FIG. 3 is a schematic block view showing main components of the toilet system shown in FIG. 1,

FIG. 4 is a schematic view showing a structure of a laser sensor of the toilet system shown in FIG. 1,

FIG. 5 is a schematic view showing a process for calculating various health indices based on measurement results of the laser sensor shown in FIG. 4,

FIG. 6 is an example of a graph showing a measurement signal of the laser sensor,

FIG. 7 is an example of a graph obtained by performing a wavelet transform on the measurement signal of the laser sensor,

FIG. 8 is an example of a graph of a pulse wave signal over time,

FIG. 9 is a flowchart showing a first operation example of the toilet system shown in FIG. 1,

FIG. 10 is a flowchart showing a second operation example of the toilet system shown in FIG. 1,

FIG. 11 is a flowchart showing a third operation example of the toilet system shown in FIG. 1,

FIG. 12 is a flowchart showing a fourth operation example of the toilet system shown in FIG. 1,

FIG. 13 is a flowchart showing a fifth operation example of the toilet system shown in FIG. 1, and

FIG. 14 is a flowchart showing a sixth operation example of the toilet system shown in FIG. 1.

### EMBODIMENTS OF INVENTION

### Structure

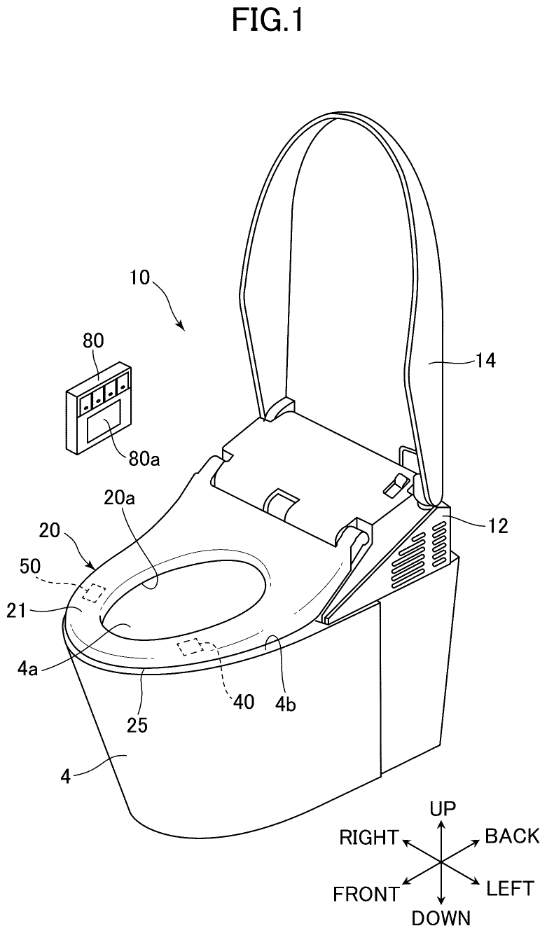

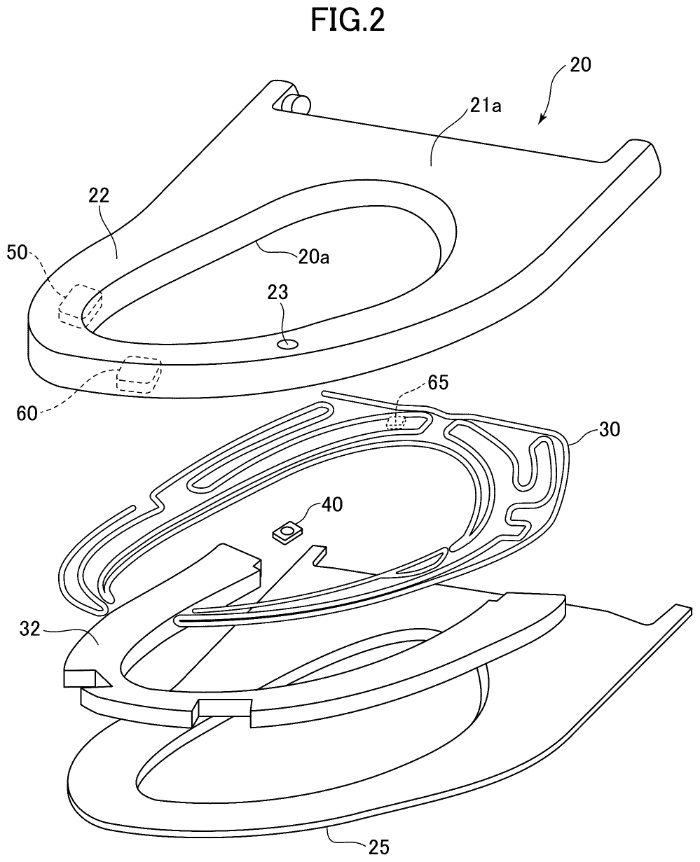

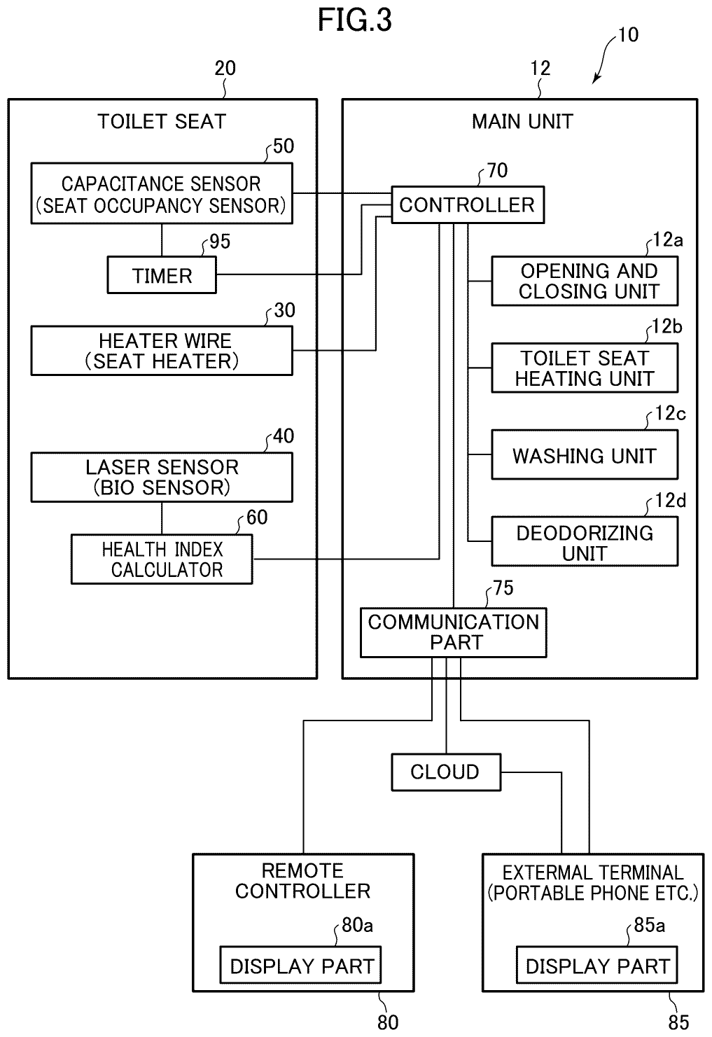

Hereinafter, an embodiment of the present invention is explained with reference to the attached drawings. FIG. 1 is a schematic perspective view of a toilet system 10 according to an embodiment of the present invention, FIG. 2 is an exploded perspective view of a toilet seat 20 of the toilet system 10 shown in FIG. 1, and FIG. 3 is a schematic block view showing main components of the toilet system 10 shown in FIG. 1.

As shown in FIGS. 1 to 3, the toilet system 10 of the present embodiment includes a toilet seat 20, a main unit 12 and a toilet cover 14. Each of the toilet seat 20 and the toilet cover 14 is supported by the main unit 12 in a pivotable manner with respect to the main unit 12.

A laser sensor 40 is provided as a sensor that measures a physical quantity which reflects blood flow information of a user. A capacitance sensor 50 is provided as a seat occupancy sensor. A health index calculator 60 (specifically, for example, a microprocessor), which is configured to calculate a plurality of health indices of the user based on measurement results by the laser sensor 40, is provided in the toilet seat 20.

The toilet seat 20 has an opening part 20a. In the present embodiment, an O-shaped opening part 20a is formed in a central area of the toilet seat 20. The opening part of the toilet seat 20 is not limited to such an O-shaped opening part, but also may be a U-shaped opening part. An outer periphery of the toilet seat 20 is curved along an outside contour of a toilet bowl unit 4. The toilet seat 20 is generally made of opaque resin (for example, polypropylene), and has a seat surface 21 on which a user is to sit, and has a bottom surface 25 opposite to the seat surface 21.

The seat surface 21 is a surface exposed upward under a state wherein the toilet seat 20 is placed on an upper surface 4b of the toilet bowl unit 4. A user is to sit on the seat surface 21. The bottom surface 25 is a surface facing to the upper surface 4b of the toilet bowl unit 4 under the same state wherein the toilet seat 20 is lowered. The substantially whole of the toilet seat 20 consists of a thick-walled portion 22, but a thin-walled portion 23, which is thinner than the thick-walled part 22, is formed locally at a position which the laser sensor 40 corresponds to.

As shown in FIG. 2, a heater wire 30 (an example of a heater) and an insulator 32 are provided in the toilet seat 20 to heat or keep warm the seat surface 21. The heater wire 30 is configured to be controlled by a toilet seat heating unit 12b provided in the main part 12 and is stretched around the inside of the toilet seat 20 so as not to interfere with the laser sensor 40, the capacitance sensor 50 and the health index calculator 60. The insulator 32 is arranged below the heater wire 30, the laser sensor 40, the capacitance sensor 50 and the health index calculator 60.

The thin-walled portion 23 is so thin that an irradiated light from the laser sensor 40 and a reflected light from the user who sits on the seat surface 21 are transmissive therethrough. The thickness of the thin-walled portion 23 is set according to intensity of the irradiated light from the laser sensor 40 and intensity of the reflected light from the user, and according to durability of the toilet seat 20, and the like. For example, the thickness of the thin-walled portion 23 is about 0.5 mm to 1.0 mm.

As used herein, the terms “up”, “down”, “front”, “back”, “left” and “right” refer to directions viewed from a user sitting on the toilet seat 20 with his or her back facing to the opened toilet cover 14, respectively.

As shown in FIG. 2, the thin-walled portion 23 is formed on the left side and on the front side than the center of the length of the opening part 20a of the toilet seat 20 in the front-back direction. That is to say, the thin-walled portion 23 is located on the left side forward from the center of gravity of a user who sits on the toilet seat 20. Thereby, the thin-walled portion 23 faces to (abuts to) a skin on a back side of the user's left thigh.

The thin-walled portion 23 is formed as small as possible to the extent that the laser sensor 40 can detect blood flow information of the user who sits on the toilet seat 20. For example, the thin-walled portion 23 has a circular shape with a diameter of 12 mm or less (preferably, 8 mm or less).

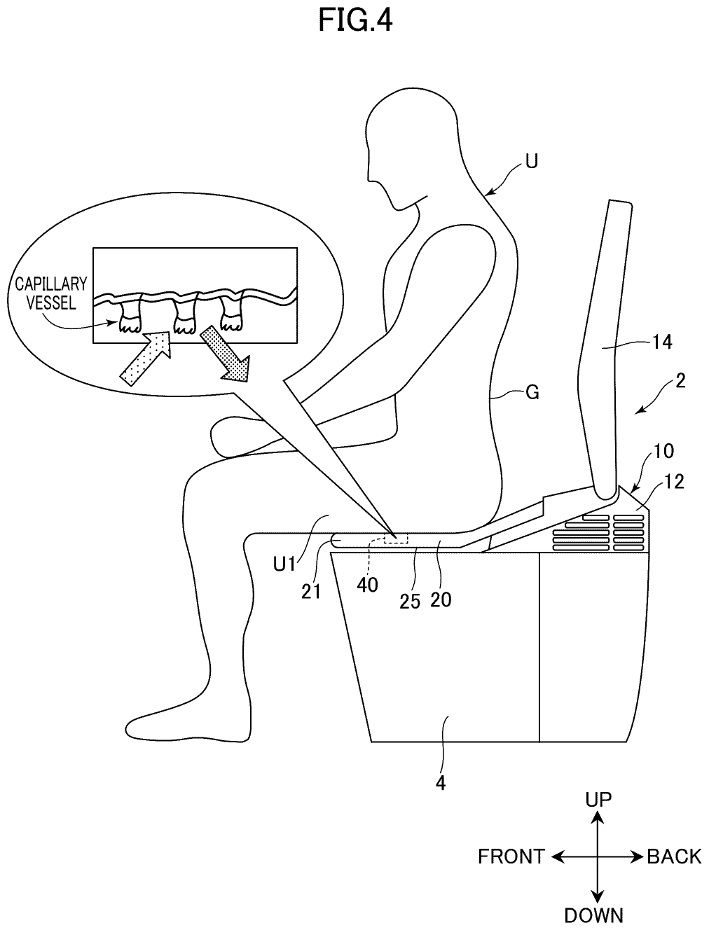

The laser sensor 40 is located on a back side of the thin-walled portion 23 in the toilet seat 20. The laser sensor 40 is a reflective type of sensor that emits an infrared irradiation light toward the back side of the user's left thigh and detects a reflected light (a scattered light resulting from Doppler Shift by red blood cells) that has been reflected according to blood flow conditions in a subcutaneous blood vessel. FIG. 4 is a schematic view showing a structure of the laser sensor 40.

On the other hand, as shown in FIGS. 1 and 2, the capacitance sensor 50 is arranged on the right side and on the front side than the center of the length of the opening part 20a of the toilet seat 20 in the front-back direction. That is to say, the capacitance sensor 50 is located on the right side forward from the center of gravity of a user who sits on the toilet seat 20. Thereby, the capacitance sensor 50 faces to (abuts to) a skin on a back side of the user's right thigh, and then the capacitance sensor 50 can detect the user's sitting state (seat occupancy state) as an example of seat occupancy sensor.

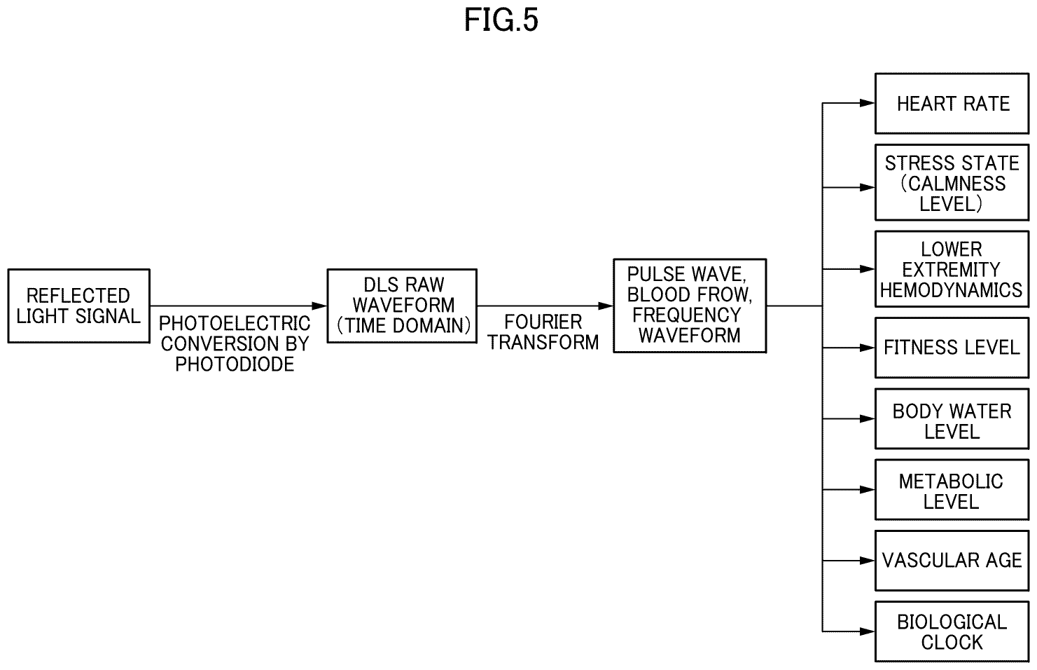

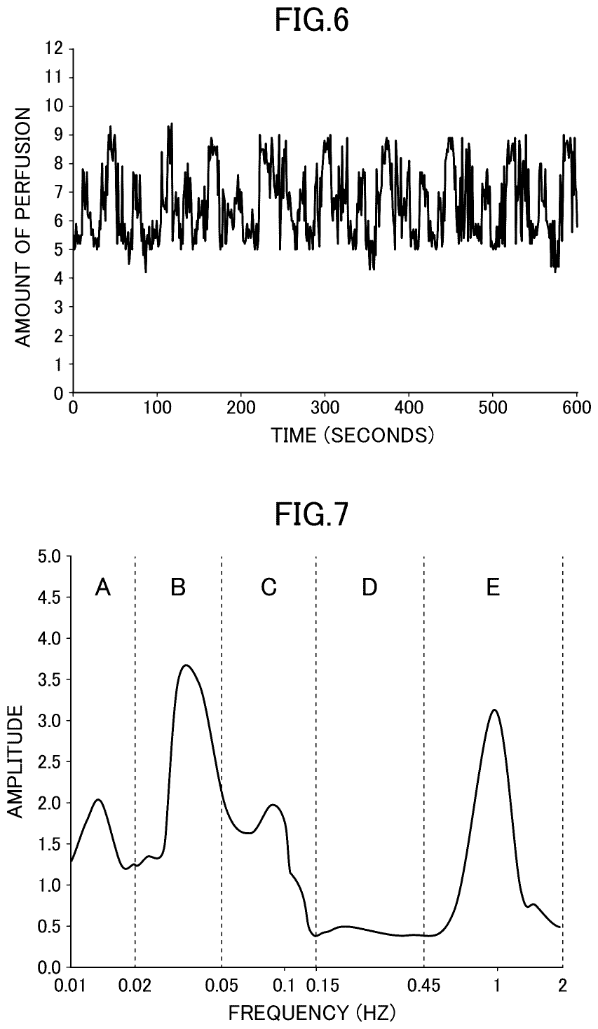

In the present embodiment, the health index calculator 60 is arranged in the vicinity of a front end of the toilet seat 20 (at a position relatively close to the laser sensor 40) and is configured to process an output signal of the laser sensor 40 and transform it to a noise-resistant signal. Specifically, the health index calculator 60 is configured to calculate a plurality of health indices (specifically, for example, a pulse rate, a pulse variability, a blood flow amount) of the user who sits on the toilet seat 20 based on the measurement results of the laser sensor 40, and to transmit a signal corresponding to the calculated results to a communication part 75 via a controller 70. FIG. 5 is a schematic view showing a process for calculating various health indices based on the measurement results of the laser sensor 40. FIG. 6 is an example of a graph showing a measurement signal of the laser sensor 40. FIG. 7 is an example of a graph obtained by performing a wavelet transform on the measurement signal of the laser sensor 40.

The controller 70 and the communication part 75 are arranged in the main unit 12. The health index calculator 60 may be arranged in the main unit 12, instead of in the toilet seat 20. When the health index calculator 60 is arranged in the main unit 12, the health index calculator 60 may be provided separately from the controller 70 or integrated with the controller 70 as a part thereof.

In addition, the health index calculator 60 and the controller 70 may be provided (set up) in an external device or an external network (for example, a cloud network) communicable via the communication part 75, instead of in the main unit 12.

With reference to FIG. 1 again, the main unit 12 is attached on the upper surface 4b of the toilet bowl unit 4 on the back side than a bowl portion of the toilet bowl unit 4. An opening and closing unit 12a configured to control an opening and losing operation for each of the toilet seat 20 and the toilet cover 14, a toilet seat heating unit 12b configured to control a temperature of the toilet seat 20, a washing unit 12c configured to wash a portion of the user's body, and a deodorizing unit 12d configured to reduce odorous components, are arranged in the main unit 12. The respective units 12a to 12d are controlled by the controller 70 comprehensively. The controller 70 is also connected to the capacitance sensor 50.

The controller 70 of the present embodiment is connected to the communication part 75 (an example of a health index output unit) for outputting the plurality of health indices of the user which has been calculated by the health index calculator 60. The communication part 75 is configured to transmit the calculated health indices of the user to, for example, a remote controller 80 in the toilet room or a portable terminal 85 of the user. Thereby, the user who sits on the toilet seat 20 can check various health indices (vital signs such as a pulse rate, etc.) by a display part 80a of the remote controller 80 and/or by a display part 85a of the portable terminal 85.

In addition, the controller 70 of the present embodiment is configured to determine whether the user is sitting on the seat surface 21 based on a measurement result of the capacitance sensor 50, as a sitting determination unit.

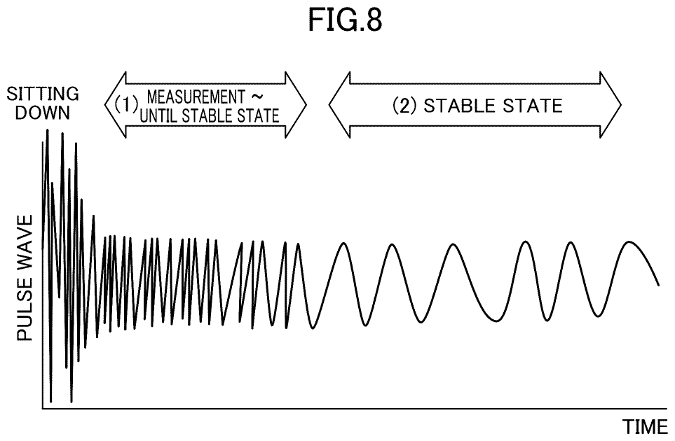

In addition, the controller 70 of the present embodiment is configured to determine whether a pulse wave signal (an example of a signal for determining a stable fluctuation, see FIG. 8) obtained by processing the measurement signal of the laser sensor 40 (for example, calculated by the health index calculator 60) has once entered a stable fluctuation state (for example, whether the user's pulse wave has once entered a stable fluctuation state), as a signal stable fluctuation determination unit.

Specifically, the controller 70 of the present embodiment is configured to determine that the pulse wave signal has once entered a stable fluctuation state when, for example, the fluctuation between time periods each of which corresponds to one cycle from a certain minimum value through a certain maximum value to the next minimum value of the pulse wave signal converges to within ±100% (a first fluctuation range) (for example, when the signal-to-noise ratio (S/N) is high, such as immediately after the user has sat down (see FIG. 6), it is highly likely that this condition will not be met), and when the fluctuation between amplitudes each of which corresponds to one cycle from a certain minimum value through a certain maximum value to the next minimum value converges to within ±20% (for example, when the signal-to-noise ratio (S/N) is high, such as immediately after the user has sat down, it is highly likely that this condition will not be met either).

Furthermore, after determining that the pulse wave signal has once entered a stable fluctuation state, the controller 70 of the present embodiment is configured to determine whether the pulse wave signal maintains such a stable fluctuation state, as a signal stable fluctuation maintenance determination unit.

Specifically, the controller 70 of the present embodiment is configured to determine that the stable fluctuation state has been interrupted (the pulse wave signal has again entered an unstable fluctuation state) when, for example, the fluctuation between time periods each of which corresponds to one cycle from a certain minimum value through a certain maximum value to the next minimum value of the pulse wave signal deviates again over ±100%. Furthermore, the controller 70 of the present embodiment is configured to determine that the stable fluctuation state has been interrupted (the pulse wave signal has entered a stable state wherein there is only little fluctuation, which means that a predetermined stable determination condition has been met) when, for example, the fluctuation between time periods each of which corresponds to one cycle from a certain minimum value through a certain maximum value to the next minimum value of the pulse wave signal converges to within ±10% (a second fluctuation range). In addition, the controller 70 of the present embodiment is configured to determine that the stable fluctuation state has been interrupted (the pulse wave signal has again entered an unstable fluctuation state) when the fluctuation between amplitudes each of which corresponds to one cycle from a certain minimum value through a certain maximum value to the next minimum value of the pulse wave signal deviates again over ±20%.

Herein, until the pulse wave signal has entered a stable fluctuation state after the user sat down on the seat surface 21, the fluctuation between time periods each of which corresponds to one cycle from a certain minimum value through a certain maximum value to the next minimum value of the pulse wave signal reduces. Therefore, it can be said that the controller 70 is configured to determine that a state within the second fluctuation range, which is smaller than the first fluctuation range, is a stable fluctuation state.

The toilet system 10 of the present embodiment has a timer 95 configured to measure a time period during which the pulse wave signal maintains a stable fluctuation state (respective time periods, if discontinuous) by cooperating with the controller 70.

As described above, an amount of the output signal from the laser sensor 40 (a measurement time by the laser sensor 40) required to calculate a health index with a certain degree of accuracy (reliability) may be different for each health index.

The toilet system 10 of the present embodiment is based on a design concept that a calculated value of the “fitness level” with a certain degree of accuracy (reliability) can be obtained when a stably fluctuating output waveform signal corresponding to a measurement time of, for example, 30 seconds (or more) is obtained.

That is to say, the controller 70 of the present embodiment is configured to determine, based on a measurement result of the capacitance sensor 50 and a measurement result of the timer 95, whether a continuous time period (or an accumulated time period, if interrupted) during which the pulse wave signal maintains a stable fluctuation state is, for example, 30 seconds or more (an example of a “predetermined total time period”: a time period between 30 seconds and 120 seconds, preferably between 30 seconds and 60 seconds, more preferably 45 seconds).

Furthermore, the timer 95 of the preset embodiment is configured to measure a time period during which the user is away from the seat surface if the user leaves the seat surface once the user has sat down on the seat surface, by cooperating with the capacitance sensor 50.

Then, in a first operation example as described below, based on the above determined results, the controller 70 is configured to cause the communication part 75 (health index output part) to: (0) stop outputting the “fitness level”, when the user leaves the seat surface before the user's pulse wave enters a stable fluctuation state, (1) output a calculated value of the “fitness level”, when the time period during which the pulse wave signal of the user maintains a stable fluctuation state is, for example, 30 seconds (an example of a “predetermined total time period”) or more after the pulse wave signal of the user has once entered the stable fluctuation state, the calculated value being based on the measurement data corresponding to the 30 seconds, and (2) stop outputting the “fitness level”, when the user leaves the seat surface before the time period during which the pulse wave signal of the user maintains a stable fluctuation state reaches, for example, 30 seconds (an example of a “predetermined total time period”) after the pulse wave signal of the user has once entered the stable fluctuation state.

Furthermore, in a second operation example as described below, based on the above determined results, the controller 70 is configured to cause the communication part 75 (health index output part) to: (3) output a calculated value of the “fitness level”, when the user leaves the seat surface temporarily (for example not more than 15 seconds) before the time period during which the pulse wave signal of the user maintains a stable fluctuation state reaches, for example, 30 seconds (an example of a “predetermined total time period”) after the pulse wave signal of the user has once entered the stable fluctuation state, and the user sits back again on the seat surface, and the time period(s) during which the pulse wave signal of the user maintains the stable fluctuation state while the user sits is accumulated to, for example, 30 seconds (an example of a “predetermined total time period”) or more, the calculated value being based on the measurement data corresponding to the 30 seconds which do not include the measurement data corresponding to one or more time periods during which the user is temporarily away from the seat surface.

Furthermore, in a third operation example as described below, based on the above determined results, the controller 70 is configured to cause the communication part 75 (health index output part) to: (4) output a calculated value of the “fitness level”, when the pulse wave signal of the user deviates from a stable fluctuation state (becomes unstable) temporarily before the time period during which the pulse wave signal of the user maintains the stable fluctuation state reaches, for example, 30 seconds (an example of a “predetermined total time period”) after the pulse wave signal of the user has once entered the stable fluctuation state, and the time period(s) during which the pulse wave signal of the user maintains the stable fluctuation state is accumulated to, for example, 30 seconds (an example of a “predetermined total time period”) or more, the calculated value being based on the measurement data corresponding to the 30 seconds which do not include the measurement data corresponding to one or more time periods during which the pulse wave signal of the user temporarily deviates from the stable fluctuation state.

Furthermore, for fourth to sixth operation examples as described below, the controller 70 of the present embodiment is configured to determine, based on a measurement result of the timer 95, whether a stable fluctuation start estimated time (set to, for example, 10 seconds after the user's sitting has been determined) at which it is estimated that the pulse wave signal of the user would have once entered a stable fluctuation state has elapsed, as a stable fluctuation estimation unit.

Then, in a fourth operation example as described below, based on the above determined results, the controller 70 is configured to cause the communication part 75 (health index output part) to: (0′) stop outputting the “fitness level”, when the user leaves the seat surface before the stable fluctuation start estimated time has elapsed after the user's sitting has been determined, (1′) output a calculated value of the “fitness level”, when the time period during which the pulse wave signal of the user maintains a stable fluctuation state is, for example, 30 seconds (an example of a “predetermined total time period”) or more after the stable fluctuation start estimated time has elapsed after the user's sitting has been determined, the calculated value being based on the measurement data corresponding to the 30 seconds, and (2′) stop outputting the “fitness level”, when the user leaves the seat surface before the time period during which the pulse wave signal of the user maintains a stable fluctuation state reaches, for example, 30 seconds (an example of a “predetermined total time period”) after the stable fluctuation start estimated time has elapsed after the user's sitting has been determined.

Furthermore, in a fifth operation example as described below, based on the above determined results, the controller 70 is configured to cause the communication part 75 (health index output part) to: (3′) output a calculated value of the “fitness level”, when the user leaves the seat surface temporarily (for example not more than 15 seconds) before the time period during which the pulse wave signal of the user maintains a stable fluctuation state reaches, for example, 30 seconds (an example of a “predetermined total time period”) after the stable fluctuation start estimated time has elapsed after the user's sitting has been determined, and the user sits back again on the seat surface, and the time period(s) during which the pulse wave signal of the user maintains (is estimated to maintain) the stable fluctuation state while the user sits is accumulated to, for example, 30 seconds (an example of a “predetermined total time period”) or more, the calculated value being based on the measurement data corresponding to the 30 seconds which do not include the measurement data corresponding to one or more time periods during which the user is temporarily away from the seat surface.

Furthermore, in a sixth operation example as described below, based on the above determined results, the controller 70 is configured to cause the communication part 75 (health index output part) to: (4′) output a calculated value of the “fitness level”, when the pulse wave signal of the user deviates from a stable fluctuation state (becomes unstable) temporarily before the time period during which the pulse wave signal of the user maintains (is estimated to maintain) the stable fluctuation state reaches, for example, 30 seconds (an example of a “predetermined total time period”) after the stable fluctuation start estimated time has elapsed after the user's sitting has been determined, and the time period(s) during which the pulse wave signal of the user maintains the stable fluctuation state is accumulated to, for example, 30 seconds (an example of a “predetermined total time period”) or more, the calculated value being based on the measurement data corresponding to the 30 seconds which do not include the measurement data corresponding to one or more time periods during which the pulse wave signal of the user temporarily deviates from the stable fluctuation state.

Herein, in the first to sixth operation examples, the toilet system is configured to output a calculated value of the “fitness level” based on a time period during which the stable fluctuation state is maintained. However, for example, the toilet system may be configured to output a calculated value of the “fitness level” based on measurement data corresponding to a predetermined time period (for example 30 seconds) during which the stable fluctuation state is maintained, going back from a time point at which the stable fluctuation state is interrupted and turns into a stable state.

In the present embodiment, the calculated and outputted “fitness level” of the user is transmitted to the remote controller 80 in the toilet room and/or the portable terminal 85 of the user. Thereby, the user who sits on the toilet seat 20 can check his or her “fitness level” on the display 80a of the remote controller 80 and/or the display 85a of the portable terminal 85.

Herein, in the present embodiment, the toilet system can output a comparison result between a “fitness level” that has been evaluated by the health index calculator 60 (an example of a fitness level evaluation part) most recently and a “fitness level” evaluated in the past.

Furthermore, in the present embodiment, the toilet system can output a comparison result between a “fitness level” that has been evaluated by the health index calculator 60 (an example of a fitness level evaluation part) most recently and a predetermined threshold value. Specifically, the “fitness level” may be evaluated as a score value between 0 and 100.

### First Operation Example: Operation

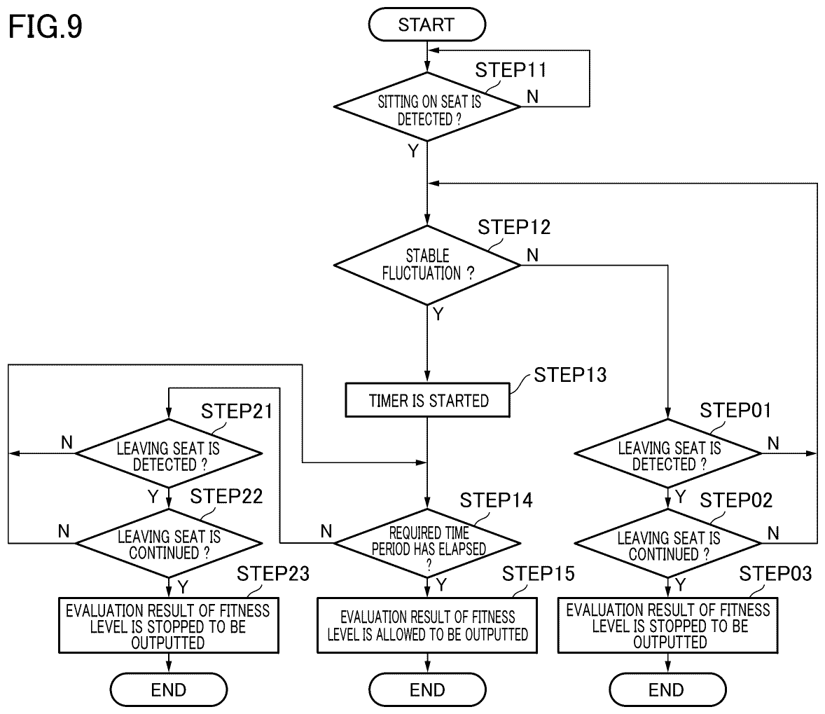

A first operation example of the toilet system 10 according to the present embodiment is explained with reference to FIG. 9. As shown in FIG. 9, the controller 70 determines whether a user sits on the seat surface 21 or not (STEP 11). When it is determined that no user has sat on the seat surface 21 yet (NO at STEP 11), this determining step is repeatedly performed.

When it is determined that a user has sat on the seat surface 21 (YES at STEP 11), the controller 70 starts to determine whether the pulse wave signal of the user (see FIG. 8) has once entered the stable fluctuation state (STEP 12). To assist in this determination, the health index calculator 60 generates (calculates) the pulse wave signal of the user based on a measurement result of the laser sensor 40.

Before the pulse wave signal of the user has once entered the stable fluctuation state (NO at STEP 12), if the capacitance sensor 50 detects that the user has left the seat surface 21 (YES at STEP 01), a seat leaving time period is measured. If the seat leaving time period is over a predetermined time period (for example, 15 seconds or less) (YES at STEP 02), the controller 70 causes the communication part 75 (health index output part) to stop outputting the evaluation result of the “fitness level” (STEP 03).

Before the pulse wave signal of the user has once entered the stable fluctuation state (NO at STEP 12), while the capacitance sensor 50 does not detect that the user has left the seat surface 21 (NO at STEP 01), it is waited for the pulse wave signal of the user to enter the stable fluctuation state (back to STEP 12).

Even if the capacitance sensor 50 detects that the user has left the seat surface 21 (YES at STEP 01), if it is determined that the user has sat again on the seat surface 21 before the seat leaving time period exceeds the predetermined time period (for example, 15 seconds or less) (NO at STEP 02), it is waited for the pulse wave signal of the user to enter the stable fluctuation state (back to STEP 12).

After the pulse wave signal of the user has once entered the stable fluctuation state (YES at STEP 12), a measurement of a certain time period by the timer 95 is started (STEP 13). For example, when 30 seconds (required time period: “predetermined total time period” in the first operation example) has elapsed (YES in STEP 14), the controller 70 causes the communication part 75 (health index output part) to output the evaluation result of the “fitness level” calculated based on the measurement data corresponding to the 30 seconds (STEP 15).

After the pulse wave signal of the user has once entered the stable fluctuation state, before the 30 seconds (required time period) has elapsed (NO in STEP 14), if the capacitance sensor 50 detects that the user has left the seat surface 21 (YES at STEP 21), a seat leaving time period is measured. If the seat leaving time period is over a predetermined time period (for example, 15 seconds or less (which value may be different from the value in the predetermined time period used for STEP 02)) (YES at STEP 22), the controller 70 causes the communication part 75 (health index output part) to stop outputting the evaluation result of the “fitness level” (STEP 23).

After the pulse wave signal of the user has once entered the stable fluctuation state, before the 30 seconds (required time period) has elapsed (NO in STEP 14), while the capacitance sensor 50 does not detect that the user has left the seat surface 21 (NO at STEP 21), it is waited for the 30 seconds (required time period) to elapse (back to STEP 14).

Even if the capacitance sensor 50 detects that the user has left the seat surface 21 (YES at STEP 21), if it is determined that the user has sat again on the seat surface 21 before the seat leaving time period exceeds the predetermined time period (for example, 15 seconds or less) (NO at STEP 22), it is waited for the 30 seconds (required time period) to elapse (back to STEP 14).

### First Operation Example: Effect

According to the first operation example as described above, measurement data from a time period before the pulse wave signal (an example of a signal for determining the stable fluctuation) has once entered the stable fluctuation state can be excluded from the evaluation target. Therefore, it is possible to obtain reliable data from the user's sitting, i.e. a normal action of the user to the toilet system, and to provide an evaluation result that more accurately reflects actual fitness level of the user.

### Second Operation Example: Operation

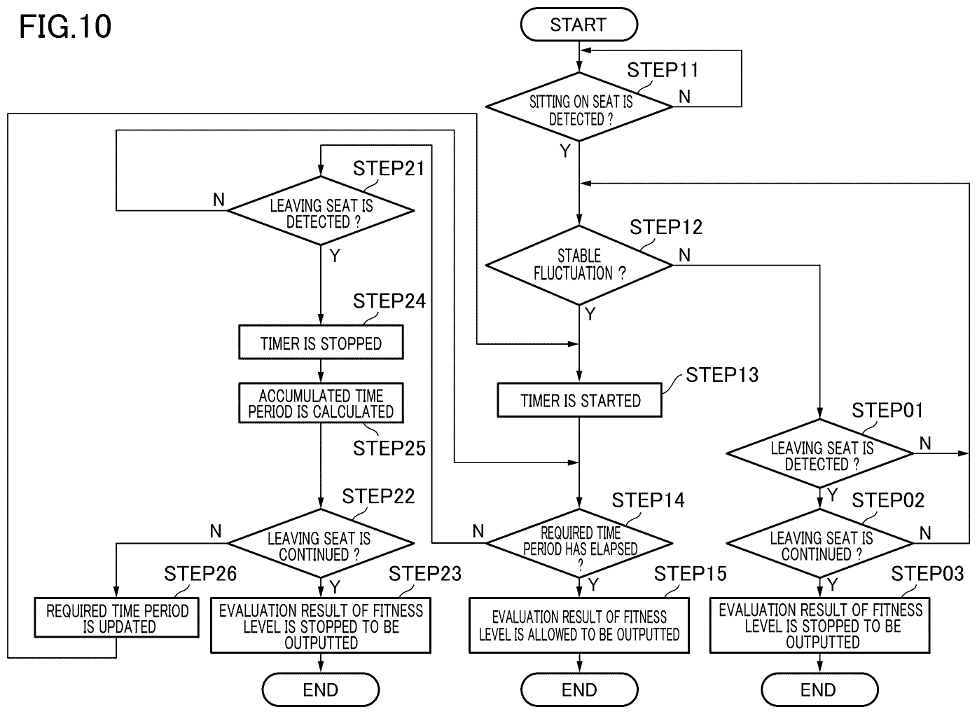

A second operation example of the toilet system 10 according to the present embodiment is explained with reference to FIG. 10. As shown in FIG. 10, the controller 70 determines whether a user sits on the seat surface 21 or not (STEP 11). When it is determined that no user has sat on the seat surface 21 yet (NO at STEP 11), this determining step is repeatedly performed.

When it is determined that a user has sat on the seat surface 21 (YES at STEP 11), the controller 70 starts to determine whether the pulse wave signal of the user (see FIG. 8) has once entered the stable fluctuation state (STEP 12). To assist in this determination, the health index calculator 60 generates (calculates) the pulse wave signal of the user based on a measurement result of the laser sensor 40.

Before the pulse wave signal of the user has once entered the stable fluctuation state (NO at STEP 12), if the capacitance sensor 50 detects that the user has left the seat surface 21 (YES at STEP 01), a seat leaving time period is measured. If the seat leaving time period is over a predetermined time period (for example, 15 seconds or less) (YES at STEP 02), the controller 70 causes the communication part 75 (health index output part) to stop outputting the evaluation result of the “fitness level” (STEP 03).

Before the pulse wave signal of the user has once entered the stable fluctuation state (NO at STEP 12), while the capacitance sensor 50 does not detect that the user has left the seat surface 21 (NO at STEP 01), it is waited for the pulse wave signal of the user to enter the stable fluctuation state (back to STEP 12).

Even if the capacitance sensor 50 detects that the user has left the seat surface 21 (YES at STEP 01), if it is determined that the user has sat again on the seat surface 21 before the seat leaving time period exceeds the predetermined time period (for example, 15 seconds or less) (NO at STEP 02), it is waited for the pulse wave signal of the user to enter the stable fluctuation state (back to STEP 12).

After the pulse wave signal of the user has once entered the stable fluctuation state (YES at STEP 12), a measurement of a certain time period by the timer 95 is started (STEP 13). Then, when a required time period which is initially for example 30 seconds (which is “predetermined total time period”-“accumulated time period”, as described below) has elapsed (YES in STEP 14), the controller 70 causes the communication part 75 (health index output part) to output the evaluation result of the “fitness level” calculated based on the measurement data corresponding to the 30 seconds (STEP 15).

After the pulse wave signal of the user has once entered the stable fluctuation state, before the required time period (initially for example 30 seconds) has elapsed (NO in STEP 14), if the capacitance sensor 50 detects that the user has left the seat surface 21 (YES at STEP 21), the measurement of the certain time period by the timer 95 is stopped (STEP 24), and the certain time period measured by the timer 95 until then (during which the pulse wave signal has maintained the stable fluctuation state) is added to an accumulated time period (which is initially 0 seconds) (STEP 25).

On the other hand, a measurement of a seat leaving time period is started. If the seat leaving time period is over a predetermined time period (for example, 15 seconds or less (which value may be different from the value in the predetermined time period used for STEP 02)) (YES at STEP 22), the controller 70 causes the communication part 75 (health index output part) to stop outputting the evaluation result of the “fitness level” (STEP 23).

After the pulse wave signal of the user has once entered the stable fluctuation state, before the required time period (initially for example 30 seconds) has elapsed (NO in STEP 14), while the capacitance sensor 50 does not detect that the user has left the seat surface 21 (NO at STEP 21), it is waited for the required time period (initially for example 30 seconds) to elapse (back to STEP 14).

Even if the capacitance sensor 50 detects that the user has left the seat surface 21 (YES at STEP 21), if it is determined that the user has sat again on the seat surface 21 before the seat leaving time period exceeds the predetermined time period (for example, 15 seconds or less) (NO at STEP 22), the required time period up to that point (initially for example 30 seconds) is updated, i.e., the accumulated time period calculated (updated) in STEP 25 is subtracted from a “predetermined total period time” (for example 30 seconds) to obtain the new required time period (STEP 26), and the timer 95 resumes measuring the measurement of the certain time period (back to STEP 13).

### Second Operation Example: Effect

According to the second operation example as described above, measurement data from a time period before the pulse wave signal (an example of a signal for determining the stable fluctuation) has first entered the stable fluctuation state can be excluded from the evaluation target. In addition, measurement data from one or more time periods during which the user is temporarily away from the toilet seat, which may be caused by the user's “body movements” while using the toilet, can be also excluded from the evaluation target. Therefore, it is possible to provide an evaluation result that even more accurately reflects actual fitness level of the user.

### Third Operation Example: Operation

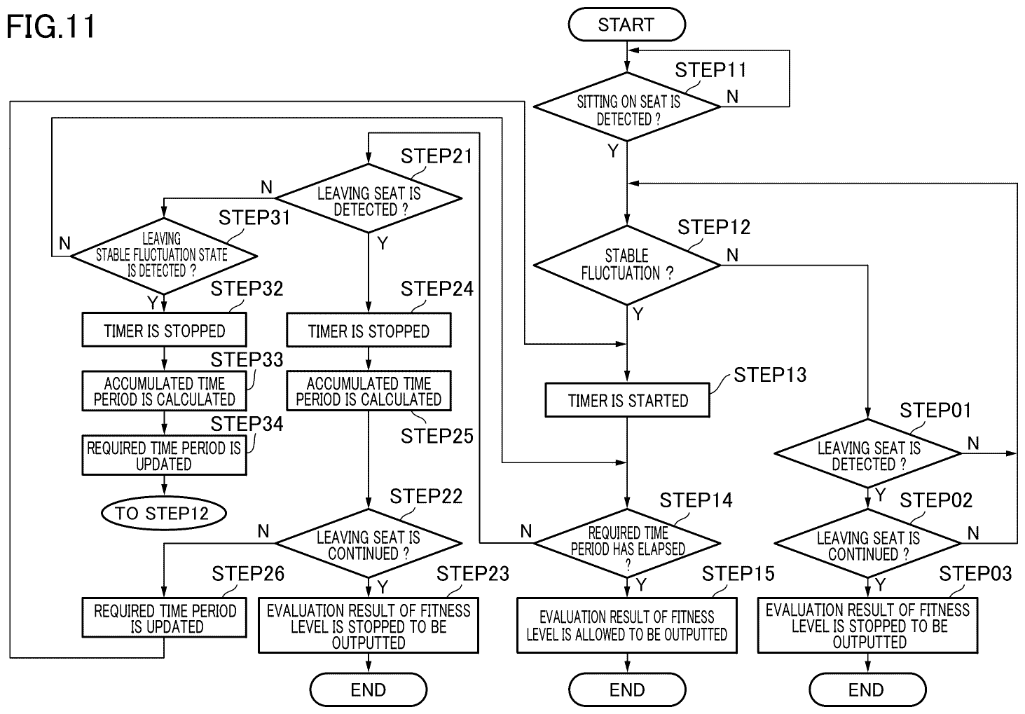

A third operation example of the toilet system 10 according to the present embodiment is explained with reference to FIG. 11. As shown in FIG. 11, the controller 70 determines whether a user sits on the seat surface 21 or not (STEP 11). When it is determined that no user has sat on the seat surface 21 yet (NO at STEP 11), this determining step is repeatedly performed.

When it is determined that a user has sat on the seat surface 21 (YES at STEP 11), the controller 70 starts to determine whether the pulse wave signal of the user (see FIG. 8) has once entered the stable fluctuation state (STEP 12). To assist in this determination, the health index calculator 60 generates (calculates) the pulse wave signal of the user based on a measurement result of the laser sensor 40.

Before the pulse wave signal of the user has once entered the stable fluctuation state (NO at STEP 12), if the capacitance sensor 50 detects that the user has left the seat surface 21 (YES at STEP 01), a seat leaving time period is measured. If the seat leaving time period is over a predetermined time period (for example, 15 seconds or less) (YES at STEP 02), the controller 70 causes the communication part 75 (health index output part) to stop outputting the evaluation result of the “fitness level” (STEP 03).

Before the pulse wave signal of the user has once entered the stable fluctuation state (NO at STEP 12), while the capacitance sensor 50 does not detect that the user has left the seat surface 21 (NO at STEP 01), it is waited for the pulse wave signal of the user to enter the stable fluctuation state (back to STEP 12).

Even if the capacitance sensor 50 detects that the user has left the seat surface 21 (YES at STEP 01), if it is determined that the user has sat again on the seat surface 21 before the seat leaving time period exceeds the predetermined time period (for example, 15 seconds or less) (NO at STEP 02), it is waited for the pulse wave signal of the user to enter the stable fluctuation state (back to STEP 12).

After the pulse wave signal of the user has once entered the stable fluctuation state (YES at STEP 12), a measurement of a certain time period by the timer 95 is started (STEP 13). Then, when a required time period which is initially for example 30 seconds (which is “predetermined total time period”-“accumulated time period”, as described below) has elapsed (YES in STEP 14), the controller 70 causes the communication part 75 (health index output part) to output the evaluation result of the “fitness level” calculated based on the measurement data corresponding to the 30 seconds (STEP 15).

After the pulse wave signal of the user has once entered the stable fluctuation state, before the required time period (initially for example 30 seconds) has elapsed (NO in STEP 14), if the capacitance sensor 50 detects that the user has left the seat surface 21 (YES at STEP 21), the measurement of the certain time period by the timer 95 is stopped (STEP 24), and the certain time period measured by the timer 95 until then (during which the pulse wave signal has maintained the stable fluctuation state) is added to an accumulated time period (which is initially 0 seconds) (STEP 25).

On the other hand, a measurement of a seat leaving time period is started. If the seat leaving time period is over a predetermined time period (for example, 15 seconds or less (which value may be different from the value in the predetermined time period used for STEP 02)) (YES at STEP 22), the controller 70 causes the communication part 75 (health index output part) to stop outputting the evaluation result of the “fitness level” (STEP 23).

Even if the capacitance sensor 50 detects that the user has left the seat surface 21 (YES at STEP 21), if it is determined that the user has sat again on the seat surface 21 before the seat leaving time period exceeds the predetermined time period (for example, 15 seconds or less) (NO at STEP 22), the required time period up to that point (initially for example 30 seconds) is updated, i.e., the accumulated time period calculated (updated) in STEP 25 is subtracted from a “predetermined total period time” (for example 30 seconds) to obtain the new required time period (STEP 26), and the timer 95 resumes measuring the measurement of the certain time period (back to STEP 13).

After the pulse wave signal of the user has once entered the stable fluctuation state, before the required time period (initially for example 30 seconds) has elapsed (NO in STEP 14), while the capacitance sensor 50 does not detect that the user has left the seat surface 21 (NO at STEP 21), if the pulse wave signal maintains the stable fluctuation state (NO at STEP 31), it is waited for the required time period (initially for example 30 seconds) to elapse (back to STEP 14).

After the pulse wave signal of the user has once entered the stable fluctuation state, before the required time period (initially for example 30 seconds) has elapsed (NO in STEP 14), while the capacitance sensor 50 does not detect that the user has left the seat surface 21 (NO at STEP 21), if the pulse wave signal becomes unstable temporarily (YES at STEP 31), the measurement of the certain time period by the timer 95 is stopped (STEP 32), the certain time period measured by the timer 95 until then (during which the pulse wave signal has maintained the stable fluctuation state) is added to the accumulated time period (which is initially 0 seconds) (STEP 33), the required time period up to that point (initially for example 30 seconds) is updated, i.e., the accumulated time period calculated (updated) in STEP 33 is subtracted from the “predetermined total period time” (for example 30 seconds) to obtain the new required time period (STEP 34), and the timer 95 resumes measuring the measurement of the certain time period (back to STEP 12).

### Third Operation Example: Effect

According to the third operation example as described above, measurement data from a time period before the pulse wave signal (an example of a signal for determining the stable fluctuation) has first entered the stable fluctuation state can be excluded from the evaluation target. In addition, measurement data from one or more time periods during which the pulse wave signal (an example of a signal for determining the stable fluctuation) deviates from the stable fluctuation state, which may be caused by the user's “straining” while using the toilet or by other “body movements”, can be also excluded from the evaluation target. Therefore, it is possible to provide an evaluation result that even more accurately reflects actual fitness level of the user.

### Fourth Operation Example: Operation

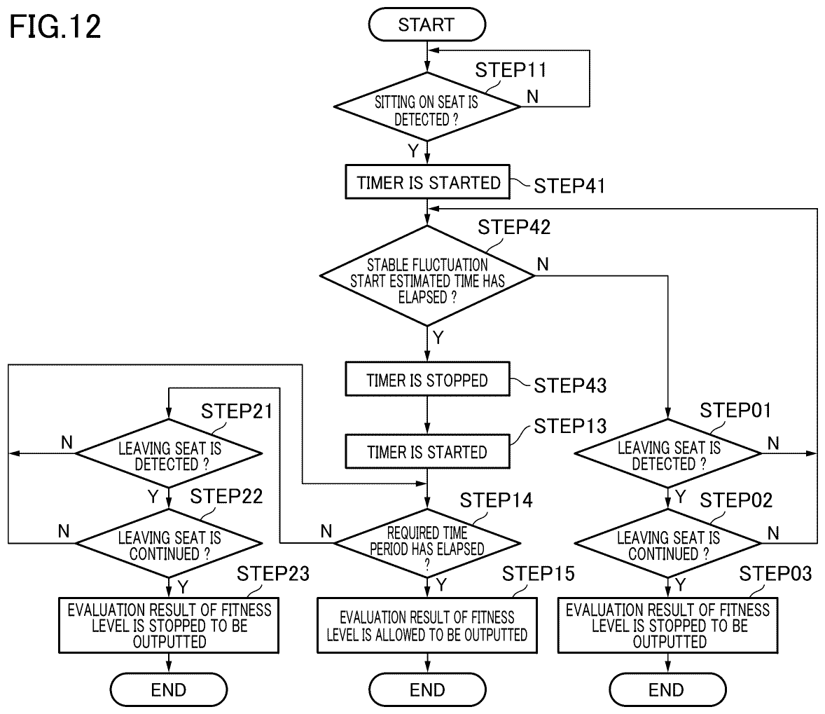

A fourth operation example of the toilet system 10 according to the present embodiment is explained with reference to FIG. 12. As shown in FIG. 12, the controller 70 determines whether a user sits on the seat surface 21 or not (STEP 11). When it is determined that no user has sat on the seat surface 21 yet (NO at STEP 11), this determining step is repeatedly performed.

When it is determined that a user has sat on the seat surface 21 (YES at STEP 11), the controller 70 starts an estimated determination as to whether it is estimated that the pulse wave signal (see FIG. 8) would have once entered a stable fluctuation state (STEP 41 to STEP 43). That is to say, a measurement of a certain time period by the timer 95 is started (STEP 41), and it is determined whether a stable fluctuation start estimated time (set to, for example, 10 seconds after the user's sitting has been determined), at which it is estimated that the pulse wave signal would have once entered a stable fluctuation state, has elapsed.

Before the stable fluctuation start estimated time has elapsed (NO at STEP 42), if the capacitance sensor 50 detects that the user has left the seat surface 21 (YES at STEP 01), a seat leaving time period is measured. If the seat leaving time period is over a predetermined time period (for example, 15 seconds or less) (YES at STEP 02), the controller 70 causes the communication part 75 (health index output part) to stop outputting the evaluation result of the “fitness level” (STEP 03).

Before the stable fluctuation start estimated time has elapsed (NO at STEP 42), while the capacitance sensor 50 does not detect that the user has left the seat surface 21 (NO at STEP 01), it is waited for the stable fluctuation start estimated time to elapse (back to STEP 42).

Even if the capacitance sensor 50 detects that the user has left the seat surface 21 (YES at STEP 01), if it is determined that the user has sat again on the seat surface 21 before the seat leaving time period exceeds the predetermined time period (for example, 15 seconds or less) (NO at STEP 02), it is waited for the stable fluctuation start estimated time to elapse (back to STEP 42).

After the stable fluctuation start estimated time has elapsed (YES at STEP 42), the timer 95 is temporarily stopped (STEP 43), and a measurement of a certain time period by the timer 95 is newly started (STEP 13). Then, when a required time period which is initially for example 30 seconds (which is “predetermined total time period”-“accumulated time period”, as described below) has elapsed (YES in STEP 14), the controller 70 causes the communication part 75 (health index output part) to output the evaluation result of the “fitness level” calculated based on the measurement data corresponding to the 30 seconds (STEP 15).

After the stable fluctuation start estimated time has elapsed (YES at STEP 42), before the 30 seconds (required time period) has elapsed (NO in STEP 14), if the capacitance sensor 50 detects that the user has left the seat surface 21 (YES at STEP 21), a seat leaving time period is measured. If the seat leaving time period is over a predetermined time period (for example, 15 seconds or less (which value may be different from the value in the predetermined time period used for STEP 02)) (YES at STEP 22), the controller 70 causes the communication part 75 (health index output part) to stop outputting the evaluation result of the “fitness level” (STEP 23).

After the stable fluctuation start estimated time has elapsed (YES at STEP 42), before the 30 seconds (required time period) has elapsed (NO in STEP 14), while the capacitance sensor 50 does not detect that the user has left the seat surface 21 (NO at STEP 21), it is waited for the 30 seconds (required time period) to elapse (back to STEP 14).

Even if the capacitance sensor 50 detects that the user has left the seat surface 21 (YES at STEP 21), if it is determined that the user has sat again on the seat surface 21 before the seat leaving time period exceeds the predetermined time period (for example, 15 seconds or less) (NO at STEP 22), it is waited for the 30 seconds (required time period) to elapse (back to STEP 14).

### Fourth Operation Example: Effect

According to the fourth operation example as described above as well, measurement data from a time period before the pulse wave signal (an example of a signal for determining the stable fluctuation) has once entered the stable fluctuation state can be excluded from the evaluation target. Therefore, it is possible to provide an evaluation result that more accurately reflects actual fitness level of the user.

In addition, according to the fourth operation example as described above, determination as to whether the predetermined sitting time has elapsed is used as a substitute for determination as to whether the pulse waver signal has once entered the stable fluctuation state. Therefore, it is possible to simplify a signal state determination routine (program).

### Fifth Operation Example: Operation

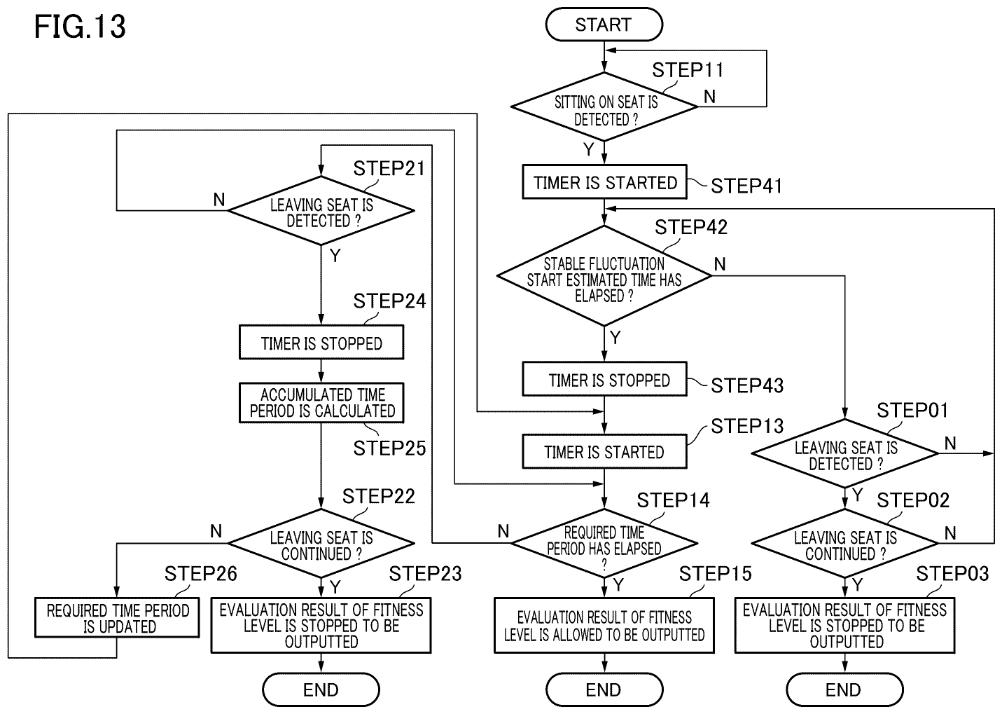

A fifth operation example of the toilet system 10 according to the present embodiment is explained with reference to FIG. 13. As shown in FIG. 13, the controller 70 determines whether a user sits on the seat surface 21 or not (STEP 11). When it is determined that no user has sat on the seat surface 21 yet (NO at STEP 11), this determining step is repeatedly performed.

When it is determined that a user has sat on the seat surface 21 (YES at STEP 11), the controller 70 starts an estimated determination as to whether it is estimated that the pulse wave signal (see FIG. 8) would have once entered a stable fluctuation state (STEP 41 to STEP 43). That is to say, a measurement of a certain time period by the timer 95 is started (STEP 41), and it is determined whether a stable fluctuation start estimated time (set to, for example, 10 seconds after the user's sitting has been determined), at which it is estimated that the pulse wave signal would have once entered a stable fluctuation state, has elapsed.

Before the stable fluctuation start estimated time has elapsed (NO at STEP 42), if the capacitance sensor 50 detects that the user has left the seat surface 21 (YES at STEP 01), a seat leaving time period is measured. If the seat leaving time period is over a predetermined time period (for example, 15 seconds or less) (YES at STEP 02), the controller 70 causes the communication part 75 (health index output part) to stop outputting the evaluation result of the “fitness level” (STEP 03).

Before the stable fluctuation start estimated time has elapsed (NO at STEP 42), while the capacitance sensor 50 does not detect that the user has left the seat surface 21 (NO at STEP 01), it is waited for the stable fluctuation start estimated time to elapse (back to STEP 42).

Even if the capacitance sensor 50 detects that the user has left the seat surface 21 (YES at STEP 01), if it is determined that the user has sat again on the seat surface 21 before the seat leaving time period exceeds the predetermined time period (for example, 15 seconds or less) (NO at STEP 02), it is waited for the stable fluctuation start estimated time to elapse (back to STEP 42).

After the stable fluctuation start estimated time has elapsed (YES at STEP 42), the timer 95 is temporarily stopped (STEP 43), and a measurement of a certain time period by the timer 95 is newly started (STEP 13). Then, when a required time period which is initially for example 30 seconds (which is “predetermined total time period”-“accumulated time period”, as described below) has elapsed (YES in STEP 14), the controller 70 causes the communication part 75 (health index output part) to output the evaluation result of the “fitness level” calculated based on the measurement data corresponding to the 30 seconds (STEP 15).

After the stable fluctuation start estimated time has elapsed (YES at STEP 42), before the required time period (initially for example 30 seconds) has elapsed (NO in STEP 14), if the capacitance sensor 50 detects that the user has left the seat surface 21 (YES at STEP 21), the measurement of the certain time period by the timer 95 is stopped (STEP 24), and the certain time period measured by the timer 95 until then (during which the pulse wave signal has maintained the stable fluctuation state) is added to an accumulated time period (which is initially 0 seconds) (STEP 25).

On the other hand, a measurement of a seat leaving time period is started. If the seat leaving time period is over a predetermined time period (for example, 15 seconds or less (which value may be different from the value in the predetermined time period used for STEP 02)) (YES at STEP 22), the controller 70 causes the communication part 75 (health index output part) to stop outputting the evaluation result of the “fitness level” (STEP 23).

After the stable fluctuation start estimated time has elapsed (YES at STEP 42), before the required time period (initially for example 30 seconds) has elapsed (NO in STEP 14), while the capacitance sensor 50 does not detect that the user has left the seat surface 21 (NO at STEP 21), it is waited for the required time period (initially for example 30 seconds) to elapse (back to STEP 14).

Even if the capacitance sensor 50 detects that the user has left the seat surface 21 (YES at STEP 21), if it is determined that the user has sat again on the seat surface 21 before the seat leaving time period exceeds the predetermined time period (for example, 15 seconds or less) (NO at STEP 22), the required time period up to that point (initially for example 30 seconds) is updated, i.e., the accumulated time period calculated (updated) in STEP 25 is subtracted from a “predetermined total period time” (for example 30 seconds) to obtain the new required time period (STEP 26), and the timer 95 resumes measuring the measurement of the certain time period (back to STEP 13).

### Fifth Operation Example: Effect

According to the fifth operation example as described above as well, measurement data from a time period before the pulse wave signal (an example of a signal for determining the stable fluctuation) has first entered the stable fluctuation state can be excluded from the evaluation target. In addition, measurement data from one or more time periods during which the user is temporarily away from the toilet seat, which may be caused by the user's “body movements” while using the toilet, can be also excluded from the evaluation target. Therefore, it is possible to provide an evaluation result that even more accurately reflects actual fitness level of the user.

In addition, according to the fifth operation example as described above, determination as to whether the predetermined sitting time has elapsed is used as a substitute for determination as to whether the pulse waver signal has once entered the stable fluctuation state. Therefore, it is possible to simplify a signal state determination routine (program).

### Sixth Operation Example: Operation

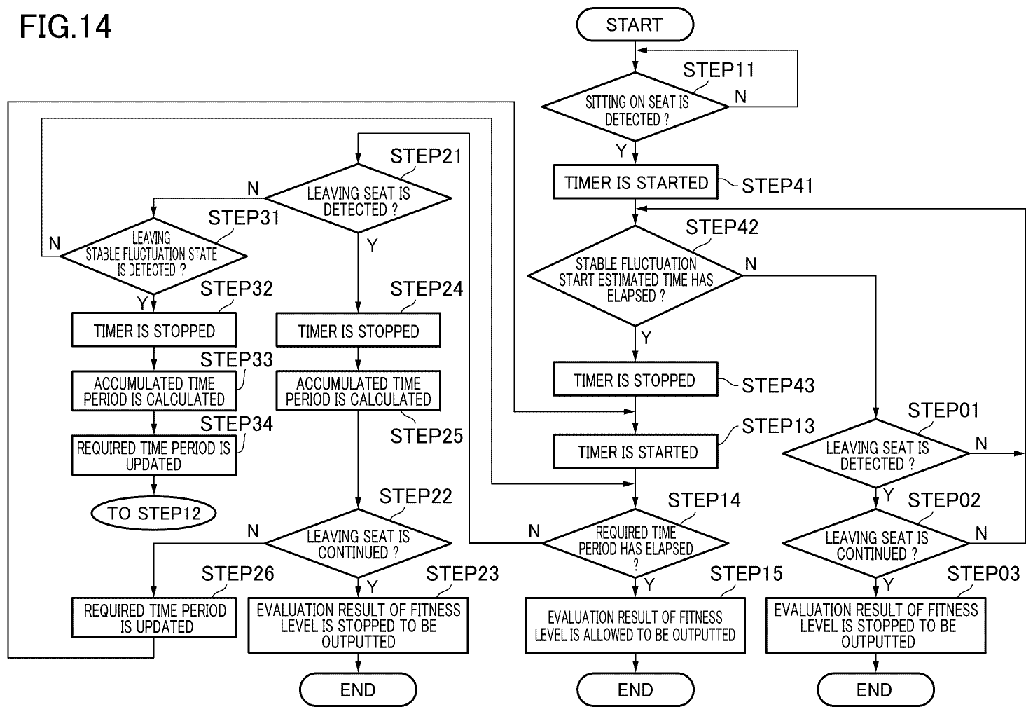

A sixth operation example of the toilet system 10 according to the present embodiment is explained with reference to FIG. 14. As shown in FIG. 14, the controller 70 determines whether a user sits on the seat surface 21 or not (STEP 11). When it is determined that no user has sat on the seat surface 21 yet (NO at STEP 11), this determining step is repeatedly performed.

When it is determined that a user has sat on the seat surface 21 (YES at STEP 11), the controller 70 starts an estimated determination as to whether it is estimated that the pulse wave signal (see FIG. 8) would have once entered a stable fluctuation state (STEP 41 to STEP 43). That is to say, a measurement of a certain time period by the timer 95 is started (STEP 41), and it is determined whether a stable fluctuation start estimated time (set to, for example, 10 seconds after the user's sitting has been determined), at which it is estimated that the pulse wave signal would have once entered a stable fluctuation state, has elapsed.

Before the stable fluctuation start estimated time has elapsed (NO at STEP 42), if the capacitance sensor 50 detects that the user has left the seat surface 21 (YES at STEP 01), a seat leaving time period is measured. If the seat leaving time period is over a predetermined time period (for example, 15 seconds or less) (YES at STEP 02), the controller 70 causes the communication part 75 (health index output part) to stop outputting the evaluation result of the “fitness level” (STEP 03).

Before the stable fluctuation start estimated time has elapsed (NO at STEP 42), while the capacitance sensor 50 does not detect that the user has left the seat surface 21 (NO at STEP 01), it is waited for the stable fluctuation start estimated time to elapse (back to STEP 42).

Even if the capacitance sensor 50 detects that the user has left the seat surface 21 (YES at STEP 01), if it is determined that the user has sat again on the seat surface 21 before the seat leaving time period exceeds the predetermined time period (for example, 15 seconds or less) (NO at STEP 02), it is waited for the stable fluctuation start estimated time to elapse (back to STEP 42).

After the stable fluctuation start estimated time has elapsed (YES at STEP 42), the timer 95 is temporarily stopped (STEP 43), and a measurement of a certain time period by the timer 95 is newly started (STEP 13). Then, when a required time period which is initially for example 30 seconds (which is “predetermined total time period”-“accumulated time period”, as described below) has elapsed (YES in STEP 14), the controller 70 causes the communication part 75 (health index output part) to output the evaluation result of the “fitness level” calculated based on the measurement data corresponding to the 30 seconds (STEP 15).

After the stable fluctuation start estimated time has elapsed (YES at STEP 42), before the required time period (initially for example 30 seconds) has elapsed (NO in STEP 14), if the capacitance sensor 50 detects that the user has left the seat surface 21 (YES at STEP 21), the measurement of the certain time period by the timer 95 is stopped (STEP 24), and the certain time period measured by the timer 95 until then (during which the pulse wave signal has maintained the stable fluctuation state) is added to an accumulated time period (which is initially 0 seconds) (STEP 25).

On the other hand, a measurement of a seat leaving time period is started. If the seat leaving time period is over a predetermined time period (for example, 15 seconds or less (which value may be different from the value in the predetermined time period used for STEP 02)) (YES at STEP 22), the controller 70 causes the communication part 75 (health index output part) to stop outputting the evaluation result of the “fitness level” (STEP 23).

Even if the capacitance sensor 50 detects that the user has left the seat surface 21 (YES at STEP 21), if it is determined that the user has sat again on the seat surface 21 before the seat leaving time period exceeds the predetermined time period (for example, 15 seconds or less) (NO at STEP 22), the required time period up to that point (initially for example 30 seconds) is updated, i.e., the accumulated time period calculated (updated) in STEP 25 is subtracted from a “predetermined total period time” (for example 30 seconds) to obtain the new required time period (STEP 26), and the timer 95 resumes measuring the measurement of the certain time period (back to STEP 13).

After the stable fluctuation start estimated time has elapsed (YES at STEP 42), before the required time period (initially for example 30 seconds) has elapsed (NO in STEP 14), while the capacitance sensor 50 does not detect that the user has left the seat surface 21 (NO at STEP 21), if the pulse wave signal maintains the stable fluctuation state (NO at STEP 31), it is waited for the required time period (initially for example 30 seconds) to elapse (back to STEP 14).

After the stable fluctuation start estimated time has elapsed (YES at STEP 42), before the required time period (initially for example 30 seconds) has elapsed (NO in STEP 14), while the capacitance sensor 50 does not detect that the user has left the seat surface 21 (NO at STEP 21), if the pulse wave signal becomes unstable temporarily (YES at STEP 31), the measurement of the certain time period by the timer 95 is stopped (STEP 32), the certain time period measured by the timer 95 until then (during which the pulse wave signal has maintained the stable fluctuation state) is added to the accumulated time period (which is initially 0 seconds) (STEP 33), the required time period up to that point (initially for example 30 seconds) is updated, i.e., the accumulated time period calculated (updated) in STEP 33 is subtracted from the “predetermined total period time” (for example 30 seconds) to obtain the new required time period (STEP 34), and the timer 95 resumes measuring the measurement of the certain time period. In FIG. 14, for the sake of simplicity, the process is shown returning to STEP 42. However, unlike the initial STEP 42 (which determines whether the stable fluctuation start estimated time has elapsed), it is actually determined at the flow position of STEP 42 whether the pulse wave signal has again entered a stable fluctuation state (this is the same determination process as STEP 31, but the YES/NO is reversed).

### Sixth Operation Example: Effect

According to the sixth operation example as described above as well, measurement data from a time period before the pulse wave signal (an example of a signal for determining the stable fluctuation) has first entered the stable fluctuation state can be excluded from the evaluation target. In addition, measurement data from one or more time periods during which the pulse wave signal (an example of a signal for determining the stable fluctuation) deviates from the stable fluctuation state, which may be caused by the user's “straining” while using the toilet or by other “body movements”, can be also excluded from the evaluation target. Therefore, it is possible to provide an evaluation result that even more accurately reflects actual fitness level of the user.

In addition, according to the sixth operation example as described above, determination as to whether the predetermined sitting time has elapsed is used as a substitute for determination as to whether the pulse waver signal has once entered the stable fluctuation state. Therefore, it is possible to simplify a signal state determination routine (program).

### First to Sixth Operation Examples: Common Effects

As described above, according to the present embodiment, it is possible to output a comparison result between a “fitness level” that has been evaluated by the health index calculator 60 (an example of a fitness level evaluation part) most recently and a “fitness level” evaluated in the past.

According to the above feature, a user of the toilet system 10 of the present embodiment can recognize his or her current fitness level each time the user uses the toilet in daily life while referring to the comparison result with the fitness level in the past.

In addition, in the present embodiment, it is possible to output a comparison result between a “fitness level” that has been evaluated by the health index calculator 60 (an example of a fitness level evaluation part) most recently and a predetermined threshold value.

According to the above feature, a user of the toilet system 10 of the present embodiment can intuitively recognize whether his or her current fitness level is good or not each time the user uses the toilet in daily life. For example, the fitness level may be evaluated as a score value between 0 and 100.

### First to Sixth Operation Examples: Variation

At STEP 03 in the above first to sixth operation examples, the evaluation result of the “fitness level” is stopped to be outputted, but instead of this manner, the controller 70 may cause the communication part 75 (health index output part) to output error information.

The error information may be outputted together with an output of the “fitness level” (which is likely to incorrectly reflect the user's actual fitness level). Alternatively, the error information may be outputted instead of (without) such an output of the “fitness level”.

Similarly, at STEP 23 as well, the evaluation result of the “fitness level” is stopped to be outputted, but instead of this manner, the controller 70 may cause the communication part 75 (health index output part) to output error information.

The error information may be outputted together with an output of the “fitness level” (which is likely to incorrectly reflect the user's actual fitness level). Alternatively, the error information may be outputted instead of (without) such an output of the “fitness level”.

In addition, at STEP 15, the evaluation result of the “fitness level” calculated based on the measurement data corresponding to the required time period (for example 30 seconds) is outputted, but instead of this manner, if a time period during which the pulse wave signal maintains a stable fluctuation state (an accumulated time period if there is one or more interruptions therebetween) is longer than the required time period (for example 30 seconds), an evaluation result of the “fitness level” calculated based on the measurement data corresponding to all that time period may be outputted, or an evaluation result of the “fitness level” calculated based on the measurement data corresponding to the required time period (for example 30 seconds) during which the pulse wave signal has entered a “more stable fluctuation state” than the rest of that time period.

Supplementary Explanation about Program

The various functions of the health index calculator 60, the controller 70 and the communication part 75 may be achieved by a microcomputer and the like which executes a corresponding program. Such a program, and a storage medium storing such a program, should be also protected by the present patent application.

For example, a program according to an aspect of the present invention is a program for providing an evaluation result of fitness level by using the toilet system 10, the toilet system including: the toilet seat 20 having the seat surface 21 on which a user is to sit; the laser sensor 40 configured to measure a physical quantity which reflects blood flow information of the user; the health index calculator 60 (a fitness level evaluation part) configured to evaluate a fitness level of the user based on a measurement result of the laser sensor 40; and the communication part 75 (a fitness level output part) configured to output the fitness level of the user which has been evaluated by the fitness level evaluation part; the program being capable of perform, when executed by a computer, the step of evaluating the fitness level of the user based on the measurement result of the laser sensor 40 that has been accumulated for one or more time periods that satisfy a predetermined condition up to a predetermined total time period.

Just in case, the present invention includes the following features (inventions).

Feature 1: A toilet system including:
a toilet seat having a seat surface on which a user is to sit;
a sensor configured to measure a physical quantity which reflects blood flow information of the user;
a fitness level evaluation part configured to evaluate a fitness level of the user based on a measurement result of the sensor; and
a fitness level output part configured to output the fitness level of the user which has been evaluated by the fitness level evaluation part;
wherein
the fitness level evaluation part is configured to evaluate the fitness level of the user based on the measurement result of the sensor that has been accumulated for one or more time periods that satisfy a predetermined condition up to a predetermined total time period.
   Feature 2: The toilet system according to feature 1, wherein
the predetermined condition is that a signal for determining a stable fluctuation based on a measurement signal of the sensor has once entered a stable fluctuation state.
   Feature 3: The toilet system according to feature 1 or 2, wherein
the predetermined condition is that a signal for determining a stable fluctuation based on a measurement signal of the sensor maintains a stable fluctuation state.
   Feature 4: The toilet system according to any of features 1 to 3, wherein
the predetermined condition is that the user has been seated on the toilet seat for more than a predetermined sitting time and a signal for determining a stable fluctuation based on a measurement signal of the sensor maintains a stable fluctuation state.
   Feature 5: The toilet system according to any of features 1 to 4, wherein
the fitness level is an index of cardiopulmonary capacity which correlates with a change in a heart rate and/or an amount of blood flow of the user.
   Feature 6: The toilet system according to any of features 1 to 5, wherein
the fitness level output part is capable of outputting a comparison result between a fitness level that has been evaluated by the fitness level evaluation part most recently and a fitness level that was evaluated by the fitness level evaluation part in the past.
   Feature 7: The toilet system according to any of features 1 to 6, wherein
the fitness level output part is capable of outputting a comparison result between a fitness level that has been evaluated by the fitness level evaluation part most recently and a predetermined threshold value.
   Feature 8: The toilet system according to any of features 1 to 7 wherein
the fitness level output part is configured not to output the fitness level of the user when the fitness level evaluation part does not evaluate the fitness level of the user based on the measurement result of the sensor that has been accumulated for the one or more time periods that satisfy the predetermined condition up to the predetermined total time period.
   Feature 9: The toilet system according to any of features 1 to 8 wherein
the sensor is an optical sensor provided in or on the toilet seat, configured to emit a light toward a leg of the user and to detect a reflected light.

### EXPLANATION OF SIGNS

4 Toilet Bowl Unit
4b Upper Surface
10 Toilet System
12 Main Unit
12a Opening and Closing Unit
12b Heating Unit
12c Washing Unit
12d Deodorizing Unit
14 Toilet Cover
20 Toilet Seat
20a Opening Part
21 Seat Surface
22 Thick-walled Portion
23 Thin-walled Portion
25 Bottom Surface
30 Heater Wire
32 Insulator
40 Laser Sensor
50 Capacitance Sensor
60 Health Index Calculator
70 Controller
75 Communication Part
80 Remote Controller
80a Display Part
85 External Terminal (Portable Phone etc.)
85a Display Part
95 Timer

What is claimed is:

1. A toilet system comprising:
a toilet seat having a seat surface on which a user is to sit;
a sensor configured to measure a physical quantity which reflects blood flow information of the user;
a fitness level evaluation part configured to evaluate a fitness level of the user based on a measurement result of the sensor; and
a fitness level output part configured to output the fitness level of the user which has been evaluated by the fitness level evaluation part;
wherein
the fitness level evaluation part is configured to evaluate the fitness level of the user based on the measurement result of the sensor that has been accumulated for one or more time periods that satisfy a predetermined condition up to a predetermined total time period.

2. The toilet system according to claim 1, wherein
the predetermined condition is that a signal for determining a stable fluctuation based on a measurement signal of the sensor has once entered a stable fluctuation state.

3. The toilet system according to claim 1, wherein
the predetermined condition is that a signal for determining a stable fluctuation based on a measurement signal of the sensor maintains a stable fluctuation state.

4. The toilet system according to claim 1, wherein
the predetermined condition is that the user has been seated on the toilet seat for more than a predetermined sitting time and a signal for determining a stable fluctuation based on a measurement signal of the sensor maintains a stable fluctuation state.

5. The toilet system according to claim 1, wherein
the fitness level is an index of cardiopulmonary capacity which correlates with a change in a heart rate and/or an amount of blood flow of the user.

6. The toilet system according to claim 5, wherein
the fitness level output part is capable of outputting a comparison result between a fitness level that has been evaluated by the fitness level evaluation part most recently and a fitness level that was evaluated by the fitness level evaluation part in the past.

7. The toilet system according to claim 5, wherein
the fitness level output part is capable of outputting a comparison result between a fitness level that has been evaluated by the fitness level evaluation part most recently and a predetermined threshold value.

8. The toilet system according to claim 5, wherein
the fitness level output part is configured not to output the fitness level of the user when the fitness level evaluation part does not evaluate the fitness level of the user based on the measurement result of the sensor that has been accumulated for the one or more time periods that satisfy the predetermined condition up to the predetermined total time period.

9. The toilet system according to claim 5, wherein
the sensor is an optical sensor provided in or on the toilet seat, configured to emit a light toward a leg of the user and to detect a reflected light.
